(19) United States
(12) Patent Application Publication
(10) Pub. No.: US 2022/0233142 A1
(43) Pub. Date: Jul. 28, 2022

(54) WEARABLE DEVICE AND METHOD OF GENERATING SIGNAL FOR CONTROLLING OPERATION OF ELECTRONIC DEVICE

(71) Applicant: Samsung Electronics Co., Ltd., Suwon-si (KR)

(72) Inventors: Raquibul HASAN, Dhaka (BD); Abdullah Al IMAM, Dhaka (BD); Asif SANJARY, Dhaka (BD); Eftekhar Hossain APU, Dhaka (BD); MD. Nayeem Jahan RAFI, Dhaka (BD); Mst. Farhana KHATUN, Dhaka (BD); Swapnil SAHA, Dhaka (BD)

(21) Appl. No.: 17/514,393

(22) Filed: Oct. 29, 2021

(30) Foreign Application Priority Data
Jan. 28, 2021 (KR) 10-2021-0012646

(51) Int. Cl. A61B 5/00; A61B 5/332; A61B 5/024; H02J 50/10; H02J 50/80; G06F 3/0488; G06F 1/16

(52) U.S. Cl. CPC A61B 5/681; A61B 5/332; A61B 5/02427; H02J 50/10; A61B 2560/04; G06F 3/0488; G06F 1/163; A61B 2560/0214; H02J 50/80

(57) ABSTRACT

A wearable device is provided. The wearable device includes a ring-shaped body having a hole, an image display unit surrounding an outer circumferential surface of the ring-shaped body, a first ring-shaped bezel and a second ring-shaped bezel respectively provided on a first side surface and a second side surface of the image display unit to be independently rotatable about the hole, a sensor configured to measure a motion, a processor configured to control a first region of the image display unit to have a touch sensing function, and control a second region that is different from the first region not to have the touch sensing function, and a transceiver unit configured to transmit, to an external electronic device, a control signal generated by the processor according to an input signal by a touch on the image display unit or an input signal by rotation of at least one of the first ring-shaped bezel or the second ring-shaped bezel.

### CROSS-REFERENCE TO RELATED APPLICATION(S)

This application is based on and claims priority under 35 U.S.C. § 119(a) of a Korean patent application number 10-2021-0012646, filed on Jan. 28, 2021, in the Korean Intellectual Property Office, the disclosure of which is incorporated by reference herein in its entirety.

### BACKGROUND

### 1. Field

The disclosure relates to a wearable device and a method of generating a signal for controlling the operation of an electronic device.

### 2. Description of Related Art

A smart watch is a well-known example of wearable devices which are designed to be worn on a part of a user's body. The smart watch may be linked with a mobile device such as a smartphone to provide a user with convenient functions such as an alarm function, a call receiving function, or a calling function. In addition, the smart watch may be conveniently worn on a wrist of the user, to measure health care information related to a heart rate, a blood pressure level, and the like of the user and provide the measured health care information to the user. As described above, the smart watch may provide useful information to the user and may be linked with a mobile device to provide a more convenient user interface environment.

Meanwhile, an electronic device, such as a television (TV), an audio set, an air conditioner (AC), or the like, generally has various switches installed at one side of a main body thereof for controlling the operation of the electronic device, and includes a remote control device such as a “remote controller” that enables remote control of the electronic device. Remote controllers are dedicated to respective electronic devices. Therefore, in order to control the operation of a target electronic device, the user has to manipulate a corresponding remote controller, and accordingly, in the case where several electronic devices are provided, it is inconvenient to individually manipulate several remote controllers respectively corresponding to the several electronic devices. As in Korean Patent Registration No. 10-0739380, wearable devices for controlling the operation of various electronic devices are disclosed.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a wearable device of a ring type which is easy to manipulate and provides a user with a more convenient user interface environment.

Another aspect of the disclosure is to provide a wearable device of a ring type capable of controlling various electronic devices via an application installed in a mobile device.

Another aspect of the disclosure is to provide a method of generating a control signal for controlling the operation of an electronic device by using a wearable device of a ring type that generates a control signal for controlling the electronic device according to a user input.

Additional aspects will be set forth in part in the description which follows and, in part, will be apparent from the description, or may be learned by practice of the presented embodiments.

In accordance with an aspect of the disclosure, a wearable device is provided. The wearable device includes a ring-shaped body including a hole, an image display unit surrounding an outer circumferential surface of the ring-shaped body, a first ring-shaped bezel and a second ring-shaped bezel respectively provided on a first side surface and a second side surface of the image display unit to be independently rotatable about the hole, a sensor configured to measure a motion, a processor configured to control a first region of the image display unit to have a touch sensing function, and control a second region that is different from the first region not to have the touch sensing function, and a transceiver unit configured to transmit, to an external electronic device, a control signal generated by the processor according to an input signal by a touch on the image display unit or an input signal by rotation of at least one of the first ring-shaped bezel or the second ring-shaped bezel.

The processor may be further configured to control an arbitrary region of the image display unit to be the first region, and control a remaining region other than the first region to be the second region based on data related to a motion measured by the sensor.

The first region may occupy about 40% to about 50% of an area of the outer circumferential surface of the image display unit.

The sensor may include at least one of a gyroscope sensor or an acceleration sensor.

The processor may be further configured to generate a first control signal when the first ring-shaped bezel and the second ring-shaped bezel are rotated in a same direction, generate a second control signal that is different from the first control signal when the first ring-shaped bezel and the second ring-shaped bezel are rotated in opposite directions, respectively, and transmit the first control signal and the second control signal to the external electronic device through the transceiver unit.

The processor may be further configured to generate a control signal corresponding to a combination of an order and angles of rotation of the first ring-shaped bezel and the second ring-shaped bezel, and transmit the control signal to the external electronic device through the transceiver unit.

The processor may be further configured to switch functions of the first ring-shaped bezel and the second ring-shaped bezel with each other based on data related to positions of the first ring-shaped bezel and the second ring-shaped bezel measured by the sensor.

The processor may be further configured to transmit, to the external electronic device, a control signal according to a touch on the first region of the image display unit, through the transceiver unit.

The first bezel and the second bezel may be implemented as images formed on both side surfaces of the image display unit, respectively, and the first ring-shaped bezel and the second ring-shaped bezel implemented as the images may be rotatable about the hole via the touch sensing function of the image display unit.

The wearable device may further include a first button on the first ring-shaped bezel and a second button on the second ring-shaped bezel.

The processor may be further configured to transmit, to the external electronic device, a first control signal based on the first button and a second control signal, which is different from the first control signal, based on the second button.

The wearable device may further include a first electrode on the first ring-shaped bezel and a second electrode on the second ring-shaped bezel.

The wearable device may further include a data processing unit configured to process an electrical signal from the first electrode and the second electrode, and the processor may be further configured to output an electrocardiogram (ECG) based on data from the data processing unit.

The wearable device may further include a photoplethysmogram (PPG) sensor on an inner circumferential surface of the ring-shaped body.

The wearable device may be configured to be rechargeable by using an external wireless charging device.

The wearable device is further configured to transmit and receive data to and from the external electronic device through the transceiver unit while charging of the wearable device is being performed by the external wireless charging device.

In accordance with another aspect of the disclosure, a method of generating a control signal for controlling operation of an electronic device by using a wearable device comprising a first mechanical rotatable structure and a second mechanical rotatable structure is provided. The method includes receiving, by the wearable device, an input signal according to rotation of the first mechanical rotatable structure and the second mechanical rotatable structure, generating, by a processor included in the wearable device, a control signal corresponding to the input signal, and transmitting, by the processor, the control signal to an external mobile device through a transceiver unit included in the wearable device.

In the generating of the control signal, the processor may be configured to generate a control signal corresponding to a combination of an order and angles of rotation of the first mechanical rotatable structure and the second mechanical rotatable structure.

The wearable device may include a ring-shaped body, and the first mechanical rotatable structure and the second mechanical rotatable structure may be respectively a first bezel and a second bezel on the body that are configured to be independently rotatable.

In the generating of the control signal, the processor may be configured to generate a first control signal when the first bezel and the second bezel are rotated in a same direction, generate a second control signal that is different from the first control signal when the first bezel and the second bezel are rotated in opposite directions, respectively, and transmit the first control signal and the second control signal to the external electronic device through the transceiver unit.

In the generating of the control signal, the processor may be configured to generate a control signal corresponding to a combination of an order and angles of rotation of the first bezel and the second bezel.

The wearable device may include a ring-shaped body, and an image display unit having a touch sensing function and surrounding an outer circumferential surface of the ring-shaped body.

The method of generating a control signal for controlling operation of an electronic device may further include receiving, by the wearable device, an input signal according to a touch on the image display unit, before the generating of, by the processor, the control signal.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating a wearable device according to an embodiment of the disclosure;

FIG. 2 is a perspective view schematically illustrating a configuration of the wearable device of FIG. 1 according to an embodiment of the disclosure;

FIG. 3 is a diagram schematically illustrating a state in which a user is wearing the wearable device of FIG. 2 according to an embodiment of the disclosure;

FIGS. 4A and 4B are diagrams schematically illustrating a configuration of image display units which may be included in the wearable device of FIG. 1 according to various embodiments of the disclosure;

FIG. 5 is a diagram illustrating a first use state of the wearable device of FIG. 1 according to an embodiment of the disclosure;

FIG. 6 is a diagram illustrating a second use state of the wearable device of FIG. 1 according to an embodiment of the disclosure;

FIG. 7 is a diagram illustrating a third use state of the wearable device of FIG. 1 according to an embodiment of the disclosure;

FIG. 8 is a diagram illustrating a fourth use state of the wearable device of FIG. 1 according to an embodiment of the disclosure;

FIG. 9 is a diagram illustrating a style matching function of the wearable device of FIG. 1 according to an embodiment of the disclosure;

FIG. 10 is a diagram schematically illustrating a use state of a first bezel which may be included in the wearable device of FIG. 2 according to an embodiment of the disclosure;

FIG. 11 is a diagram illustrating another use state of the first bezel of FIG. 10 according to an embodiment of the disclosure;

FIG. 12 is a perspective view schematically illustrating a configuration of a wearable device according to an embodiment of the disclosure;

FIG. 13 is a diagram illustrating an electrocardiogram (ECG) measurement system which may be included in the wearable device of FIG. 12 according to an embodiment of the disclosure;

FIG. 14 is a diagram illustrating a use state of the ECG measurement system of FIG. 13 according to an embodiment of the disclosure;

FIG. 15 is a diagram illustrating a photoplethysmogram sensor which may be included in the wearable device of FIG. 2 according to an embodiment of the disclosure;

FIG. 16 is a flowchart of a method of generating a signal for controlling an electronic device, according to an embodiment of the disclosure;

FIG. 17 is a diagram schematically illustrating a configuration of an electronic device control system according to an embodiment of the disclosure;

FIG. 18 is a flowchart of a method of controlling the operation of an electronic device, according to an embodiment of the disclosure;

FIG. 19 is a diagram illustrating an operation state of the electronic device control system of FIG. 17 according to an embodiment of the disclosure;

FIG. 20 is a diagram illustrating another operation state of the electronic device control system of FIG. 17 according to an embodiment of the disclosure;

FIG. 21 is a flowchart of a method of unlocking a mobile device by using the wearable device of FIG. 2 according to an embodiment of the disclosure;

FIG. 22 is a diagram illustrating a method of performing wireless charging of the wearable device of FIG. 17 according to an embodiment of the disclosure;

FIG. 23 is a diagram illustrating a method of performing wireless charging of the wearable device of FIG. 17 according to an embodiment of the disclosure;

FIGS. 24A and 24B are diagrams illustrating an operation of the mobile device of FIG. 23 while performing wireless charging of the wearable device of FIG. 23 according to various embodiments of the disclosure;

FIG. 25 is a diagram illustrating an interaction between the wearable device of FIG. 17 and an external electronic device that occurs while performing wireless charging of the wearable device according to an embodiment of the disclosure;

FIG. 26 is a diagram illustrating a method of performing wireless charging of the wearable device of FIG. 17 according to an embodiment of the disclosure; and

FIG. 27 is a diagram illustrating an interaction between the wearable device of FIG. 26 and an electronic device that occurs while performing wireless charging of the wearable device according to an embodiment of the disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Throughout the disclosure, the expression “at least one of a, b or c” indicates only a, only b, only c, both a and b, both a and c, both b and c, all of a, b, and c, or variations thereof.

Hereinafter, a wearable device and a method of generating a signal for controlling the operation of an electronic device according to various embodiments of the disclosure will be described in detail with reference to the accompanying drawings. In the drawings, like reference numerals refer to like elements, and the size or thickness of each element may be exaggerated for clarity of description.

Terms such as “first” or “second” may be used to describe various elements, but the elements should not be limited by the terms. These terms are only used to distinguish one element from another element. The wearable device and the method of generating a signal for controlling the operation of an electronic device may be embodied in many different forms and should not be construed as being limited to the embodiments of the disclosure set forth herein.

Throughout the specification, when an element is referred to as “including” a constituent element, other constituent elements may be further included not excluded unless there is any other particular mention on it.

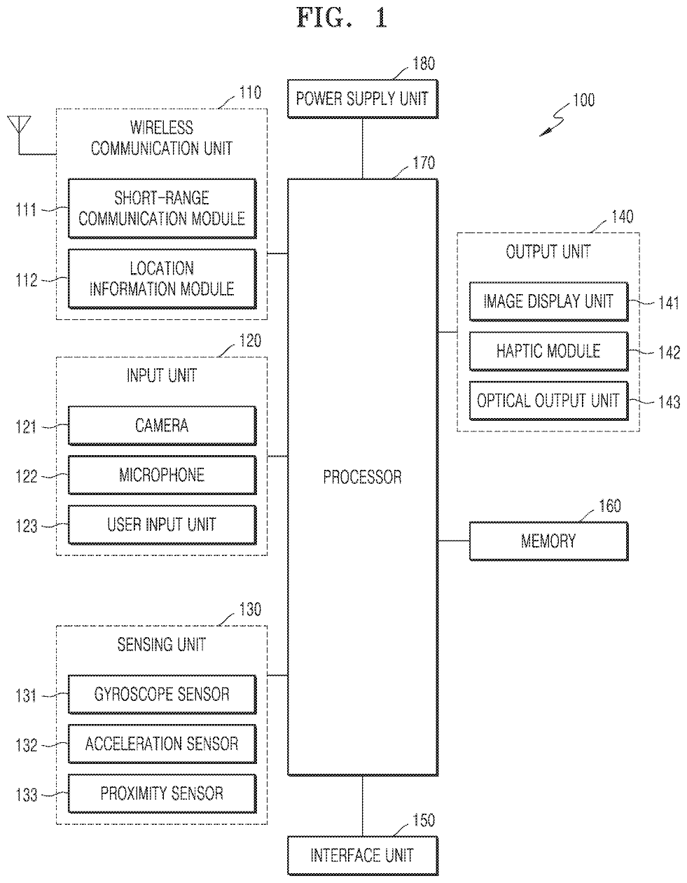

FIG. 1 is a block diagram illustrating a wearable device 100 according to an embodiment of the disclosure.

Referring to FIG. 1, the wearable device 100 may include a wireless communication unit 110 (transceiver unit), an input unit 120, a sensing unit 130, an output unit 140, an interface unit 150, a memory 160, a processor 170, and a power supply unit 180. The components illustrated in FIG. 1 are not essential in implementing the wearable device 100, and thus, the wearable device 100 may have more or fewer components than the components listed above.

The wireless communication unit 110 (transceiver unit) may include a short-range communication module 111 and a location information module 112. Although not shown in FIG. 1, the wireless communication unit 110 may further include a broadcast receiving module (not shown) for receiving a broadcast signal and/or broadcast-related information from an external broadcast management server through a broadcast channel such as a satellite channel, a terrestrial channel, or the like, a mobile communication module (not shown) for transmitting and receiving a wireless signal to and from a base station, an external terminal, a server, or the like over a mobile communication network established by using a standard communication technology, and a wireless Internet module (not shown) for transmitting and receiving a wireless signal in a communication network according to various wireless Internet technologies such as Wireless-Fidelity (Wi-Fi), WiBro, Long-Term Evolution (LTE), and the like.

The short-range communication module 111 is for short-range communication, and may support short-range communication by using at least one of Bluetooth™, radio frequency identification (RFID), Infrared Data Association (IrDA), ultra-wideband (UWB), ZigBee, near-field communication (NFC), Wireless-Fidelity (Wi-Fi), Wi-Fi Direct, or wireless universal serial bus (USB). The short-range communication module 111 may support wireless communication between the wearable device 100 and a wireless communication system, between the wearable device 100 and another external electronic device, or between the wearable device 100 and a network to which another external electronic device (or an external server) is connected, through a wireless local area network. The wireless local area network may be a wireless personal area network.

The location information module 112 is a module for obtaining location information of the wearable device 100, and representative examples thereof include a global positioning system (GPS) module or a Wireless Fidelity (Wi-Fi) module. For example, by using a GPS module, the location information of the wearable device 100 may be obtained by using a signal transmitted from a GPS satellite. As another example, by using a Wi-Fi module, the location information of the wearable device 100 may be obtained based on information of a wireless access point (AP) that transmits or receives a wireless signal to or from the Wi-Fi module. The location information module 112 is a module used for obtaining the location information of the wearable device 100, and is not limited to a module that directly calculates or obtains the location of the wearable device 100.

The input unit 120 is for receiving image information (or an image signal), audio information (or an audio signal), data, or information that is input by a user. The input unit 120 may include one or more cameras 121 for receiving image information. The camera 121 may process an image frame such as a still image or a moving image obtained by an image sensor in a video call mode or an image capture mode. The processed image frame may be displayed on an image display unit 141 which will be described below, or may be stored in the memory 160.

The one or more cameras 121 included in the input unit 120 may be arranged to constitute a matrix structure, and a plurality of pieces of image information having various angles or focuses may be input to the wearable device 100 through the one or more cameras 121 constituting the matrix structure. In addition, the one or more cameras 121 may be arranged in a stereo structure to obtain a left image and a right image for implementing a stereoscopic image.

In addition, the input unit 120 may include a microphone 122 for receiving audio information. The microphone 122 processes an external sound signal into electrical audio data. The obtained audio data may be variously utilized according to a function (or an application program being executed) being performed by the wearable device 100. Meanwhile, various denoising algorithms may be performed by the microphone 122 to remove noise generated when receiving the external sound signal.

Furthermore, the input unit 120 may include a user input unit 123 for receiving information from the user. When information is input through the user input unit 123, the processor 170 may generate a control signal corresponding to the input information. The user input unit 123 may include a mechanical input member and a touch input member.

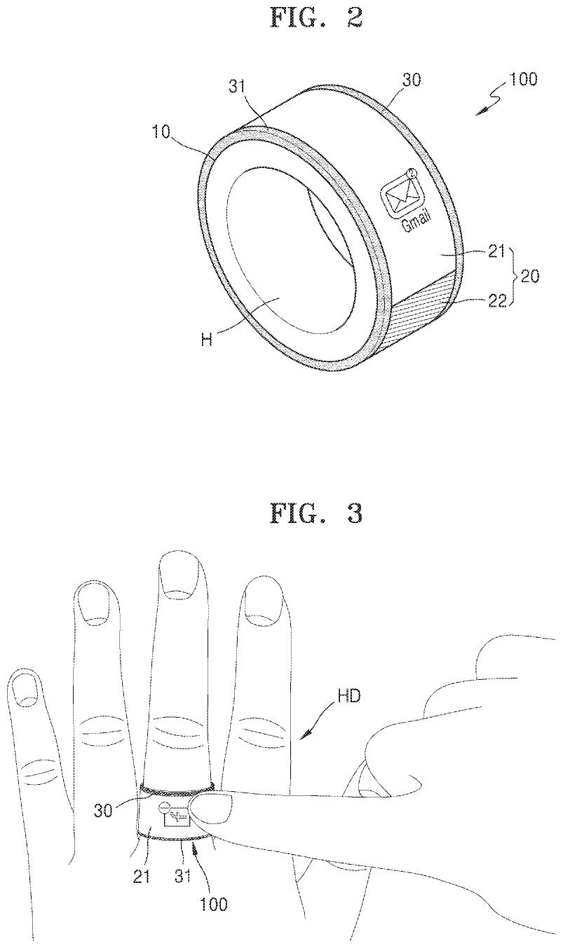

For example, the mechanical input member may include a first bezel 30 and a second bezel 31 of FIG. 2, which will be described below. The first bezel 30 and the second bezel 31 are configured to be rotatable, and various input signals may be input to the wearable device 100 according to various combinations of angles and directions of rotation of the first bezel 30 (e.g., a first mechanical rotatable structure) and the second bezel 31 (e.g., a second mechanical rotatable structure). Configurations of the first bezel 30 and the second bezel 31 will be described with reference to FIG. 2.

The touch input member may include a virtual key, a soft key, or a visual key displayed on the image display unit 141 which will be described below, through software processing. Meanwhile, the virtual key or the visual key may be displayed on the image display unit 141 in various forms, and may be composed of, for example, graphics, texts, icons, videos, or a combination thereof.

The sensing unit 130 may include one or more sensors for sensing at least one of surrounding environment information of the wearable device 100 or user information. For example, the sensing unit 130 may include a gyroscope sensor 131 for measuring a motion of the user, an acceleration sensor 132, a proximity sensor 133, and the like. Furthermore, although not shown in FIG. 1, the sensing unit 130 may include at least one of an illumination sensor, a magnetic sensor, a gravity sensor (G-sensor), a motion sensor, a red, green, blue (RGB) sensor, an infrared sensor (IR sensor), a finger scan sensor, an ultrasonic sensor, an optical sensor, a battery gauge, an environmental sensor (e.g., a barometer, a hygrometer, a thermometer, a radiation sensor, a heat sensor, a gas sensor, and the like), or a chemical sensor (e.g., an electronic nose, a healthcare sensor, a biometric sensor, and the like). The wearable device 100 may combine and utilize pieces of information sensed by at least two of the sensors.

The output unit 140 is for generating an output related to a visual, auditory, or tactile sense. For example, the output unit 140 may include the image display unit 141. Also, the output unit 140 may further include a haptic module 142, a light output unit 143, and the like.

The image display unit 141 may be implemented as a touch screen by constituting an interlayer structure with a touch sensor, or by being integrally formed with a touch sensor. The touch screen may function as the user input unit 123 that provides an input interface between the wearable device 100 and the user, and may also provide an output interface between the wearable device 100 and the user.

The haptic module 142 produces various tactile effects. Representative examples of the tactile effects produced by the haptic module 142 may include a vibration effect. The intensity and pattern of a vibration generated by the haptic module 142 may be controlled by the user's selection or a setting of the processor 170. For example, the haptic module 142 may output a combination of different vibrations or sequentially output different vibrations.

In addition to the vibration effect, the haptic module 142 may produce a variety of tactile effects such as an effect by stimulation due to the arrangement of pins moving perpendicular to a skin contact surface, an effect by stimulation by the jetting force or the suction force of air through an injection nozzle or an inlet, an effect by stimulation by grazing a skin surface, an effect by stimulation through contact of an electrode, an effect by stimulation by an electrostatic force, an effect by reproducing sense of coldness or warmth by using an element capable of absorbing or emitting heat, and the like.

The haptic module 142 may provide a tactile effect through direct contact, or may allow the user to feel a tactile effect through a muscle sensation in a finger or arm of the user. Two or more haptic modules 142 may be provided according to a configuration aspect of the wearable device 100.

The light output unit 143 may output a signal for notifying that an event of the wearable device 100 has occurred by using light of a light source. Examples of the event occurring in the wearable device 100 may include message reception, signal reception, a missed call, an alarm, schedule notification, email reception, information reception by an application, and the like. The signal output by the light output unit 143 is implemented as the wearable device 100 emits light of a single color or a plurality of colors from the front or rear of the wearable device 100. Outputting of a signal by the light output unit 143 may be terminated when the wearable device 100 detects that the user has confirmed the event.

The interface unit 150 may serve as a path to all external electronic devices connected to the wearable device 100. The interface unit 150 may receive data or power from the external electronic devices, to transmit the received data or power to each component in the wearable device 100, or transmit internal data of the wearable device 100 to an external device. For example, the interface unit 150 may include a wired/wireless headset port, an external charger port, a wired/wireless data port, a memory card port, a port for connection with a device having an identification module, an audio input/output (I/O) port, a video I/O port, an earphone port, and the like.

The memory 160 may store a program for the operation of the processor 170 and may temporarily store input or output data. The memory 160 may store data related to vibrations and sounds having various patterns that are output when a touch is input to the touch screen.

The memory 160 may include at least one of a flash memory-type storage medium, a hard disk-type storage medium, a solid-state disk (SSD)-type storage medium, a silicon disk drive (SDD)-type storage medium, a multimedia card micro-type storage medium, card-type memory (e.g., SD or XD memory), random access memory (RAM), static random-access memory (SRAM), read-only memory (ROM), electrically erasable programmable read-only memory (EEPROM), programmable read-only memory (PROM), magnetic memory, a magnetic disc, or an optical disc.

The processor 170 generally controls operations related to an application program and overall operations of the wearable device 100. In addition, the processor 170 may process data input to the input unit 120, or perform pattern recognition processing to recognize characters or images respectively from a handwriting input or a drawing input received via the touch screen implemented by the image display unit 141. Furthermore, in order to implement various embodiments described below in the wearable device 100, the processor 170 may control any one or a combination of the components described above.

The power supply unit 180 may be a device for supplying electric energy required for the operation of the wearable device 100. For example, the power supply unit 180 may include a battery. The power supply unit 180 may be a built-in battery designed to be rechargeable, and may be detachably coupled to the wearable device 100 for charge thereof, and the like.

The power supply unit 180 may include a connection port, and the connection port may be configured as an example of the interface unit 150 to which an external charging device that supplies power for charging the battery is electrically connected.

As another example, the power supply unit 180 may be configured to charge the battery in a wireless manner without using the connection port. In this case, the power supply unit 180 may receive power from an external wireless charging device by using at least one of an inductive coupling scheme based on magnetic induction, or a magnetic resonance coupling scheme based on electromagnetic resonance. Accordingly, the wearable device 100 may be configured to be rechargeable by using the external wireless charging device.

FIG. 2 is a perspective view schematically illustrating a configuration of the wearable device 100 of FIG. 1 according to an embodiment of the disclosure.

FIG. 3 is a diagram schematically illustrating a state in which a user is wearing the wearable device 100 of FIG. 2 according to an embodiment of the disclosure.

Referring to FIG. 2, the wearable device 100 may include a body 10 of a ring type including a hole H into which a finger of the user is to be inserted, an image display unit 20 surrounding an outer circumferential surface of the body 10, and the first and second bezels 30 and 31 of ring types respectively on both side surfaces of the image display unit 20 to be independently rotatable about the hole H. The image display unit 20 may include a touch sensing function. The image display unit 20 may be substantially the same as the image display unit 141 included in the output unit 140 of FIG. 1.

In addition, the wearable device 100 may further include the processor 170 configured to control a first region 21 of the image display unit 20 to have the touch sensing function, and a second region 22 different from the first region 21 not to have the touch sensing function. In addition, the wearable device 100 may further include the wireless communication unit 110 configured to transmit, to an external electronic device, a control signal generated by the processor 170 according to an input signal based on a touch on the image display unit 20 or an input signal based on rotation of at least one of the first bezel 30 or the second bezel 31.

The body 10 may have a hollow cylindrical shape including the hole H having a diameter sufficient to fit the finger of the user. However, the disclosure is not limited thereto, and the body 10 may have various hollow column shapes other than the hollow cylindrical shape. For example, the body 10 may have an integral three-dimensional shape including an inner circumferential surface to surround the finger of the user and an outer circumferential surface.

The image display unit 20 may show the user an image containing various pieces of information. For example, the image display unit 20 may include a liquid crystal display (LCD), an organic light-emitting diode (OLED) display, and the like. However, the disclosure is not limited thereto, and the image display unit 20 may include various types of devices that generate image information. The image displayed on the image display unit 20 may be variously produced according to an electrical signal transmitted from a processor. In addition, the image display unit 20 may have a curved configuration with sufficient flexibility to surround the outer circumferential surface of the body 10.

Meanwhile, the image display unit 20 may include the first region 21 having the touch sensing function and the second region 22 not having the touch sensing function. A clear boundary line may be formed between the first region 21 and the second region 22. The user may clearly recognize the first region 21 having the touch sensing function through the boundary line formed between the first region 21 and the second region 22. In addition, because only a portion (the first region 21) of the image display unit 20 has the touch sensing function, unintended touch inputs due to mistakes of the user may be reduced.

The processor 170 may transmit, to the external electronic device, a control signal according to a touch on the first region 21 of the image display unit 20, through the wireless communication unit 110. For example, the processor 170 may generate various control signals according to various types of touches, such as a tap, a hard tap, a double tap, a short touch, a long touch, a multi touch, a drag touch, a complete cover, and the like, on the first region 21 of the image display unit 20, and may transmit the generated control signals to the external electronic device.

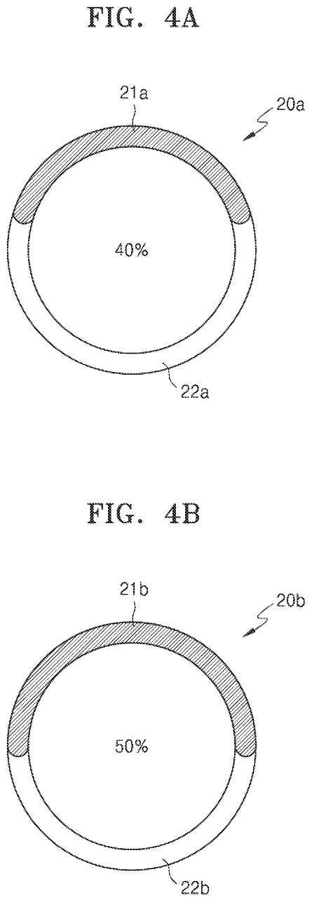

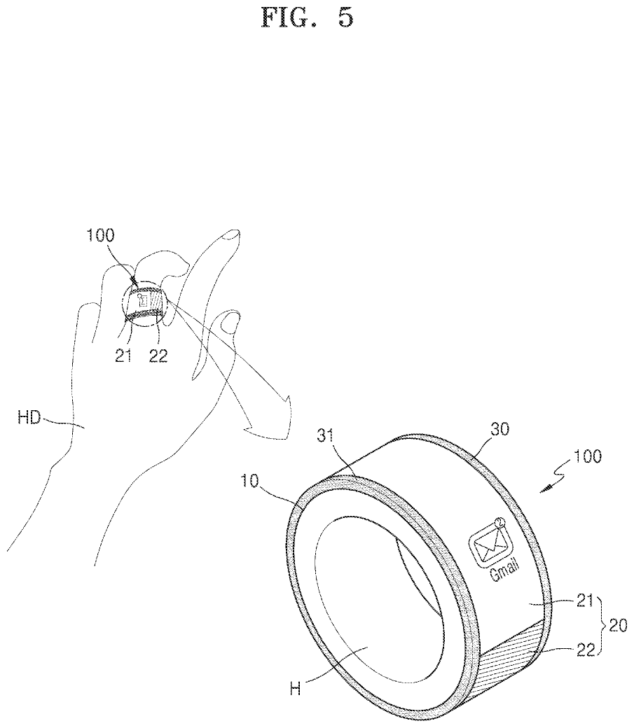

The processor 170 may control an arbitrary region of the image display unit 20 to be the first region 21 and control the remaining region other than the first region 21 to be the second region 22 based on data related to motions of the user, measured by the gyroscope sensor 131, the acceleration sensor 132, and the proximity sensor 133 included in the sensing unit 130. A method, performed by the processor 170, of dividing the image display unit 20 into the first region 21 and the second region 22 will be described with reference to FIGS. 4 to 8.

The first bezel 30 and the second bezel 31 may be a ring type formed to be rotatable about the hole H of the body 10. As illustrated in FIG. 2, the first bezel 30 and the second bezel 31 may be provided respectively on both side surfaces of the image display unit 20 to surround a portion of the outer circumferential surface of the body 10. The first bezel 30 and the second bezel 31 may be provided respectively at edges of the outer circumferential surface of the body 10. Accordingly, the first bezel 30 and the second bezel 31 may be provided on the outer circumferential surface of the body 10 to be apart from each other with the image display unit 20 therebetween. The first bezel 30, the image display unit 20, and the second bezel 31 may be sequentially provided to form one hollow column.

The first bezel 30 and the second bezel 31 may be provided in parallel to each other, and may be configured to rotate independently of each other. Accordingly, the first bezel 30 and the second bezel 31 may be rotated in the same direction or in different directions. When the user rotates the first bezel 30 and the second bezel 31, an input signal may be input to the wearable device 100. In addition, when the first bezel 30 and the second bezel 31 are rotated, a certain electrical signal is generated, and the processor 170 processes the electrical signal to generate a control signal. The processor 170 may transmit, to the external electronic device, the control signal generated according to the rotation of at least one of the first bezel 30 and the second bezel 31, through the short-range communication module 111 included in the wireless communication unit 110. The processor 170 may generate a control signal corresponding to a combination of an order and angles of rotation of the first bezel 30 and the second bezel 31.

For example, the processor 170 may generate a first control signal when the first bezel 30 and the second bezel 31 are rotated in the same direction. In addition, the processor 170 may generate a second control signal that is different from the first control signal when the first bezel 30 and the second bezel 31 are rotated in opposite directions, respectively.

As described above, the user may control the operation of the external electronic device by appropriately adjusting the rotation of the first bezel 30 and the second bezel 31. A method of controlling the external electronic device by rotating the first bezel 30 and the second bezel 31 will be described with reference to FIGS. 17 to 20.

Referring to FIG. 3, the user wearing the wearable device 100 on a hand HD may provide an input signal to the wearable device 100 by touching the first region 21, which has the touch sensing function, of the image display unit 20 or by rotating the first bezel 30 and the second bezel 31, with the other hand.

Meanwhile, the processor 170 may switch functions of the first bezel 30 and the second bezel 31 with each other based on data related to positions of the first bezel 30 and the second bezel 31 measured by the gyroscope sensor 131, the acceleration sensor 132, and the proximity sensor 133 included in the sensing unit 130. For example, when the user is wearing the wearable device 100 on the finger, the processor 170 may control the bezel closer to the palm to have a first function and the other bezel to have a second function.

For example, in the case where the user is wearing the wearable device 100 such that the first bezel 30 is closer to the palm than the second bezel 31, the processor 170 may generate the first control signal when the first bezel 30 is rotated, and generate the second control signal when the second bezel 31 is rotated. On the contrary, in the case where the user is wearing the wearable device 100 such that the second bezel 31 is closer to the palm than the first bezel 30, the processor 170 may generate the first control signal when the second bezel 31 is rotated, and generate the second control signal when the first bezel 30 is rotated.

FIGS. 4A and 4B are diagrams schematically illustrating a configuration of image display units 20a and 20b, which may be included in the wearable device 100 of FIG. 1 according to various embodiments of the disclosure.

Referring to FIGS. 4A and 4B, the image display units 20a and 20b, which may be included in the wearable device 100 of FIG. 1, may include first regions 21a and 21b having the touch sensing function and second regions 22a and 22b not having the touch sensing function, respectively. The image display units 20a and 20b may include touch electrodes for implementing the touch sensing function, that are provided in a region corresponding to the entire outer circumferential surface. However, the processor 170 may control some of the touch electrodes included in the image display units 20a and 20b not to operate, and thus the second regions 22a and 22b not having the touch sensing function may be implemented. For example, as illustrated in FIG. 4A, the first region 21a may occupy 40% of the area of the outer circumferential surface of the image display unit 20a. Also, as illustrated in FIG. 4B, the first region 21b may occupy 50% of the area of the outer circumferential surface of the image display unit 20b. Furthermore, the first regions 21a and 21b may occupy about 40% to about 50% of the area of the outer circumferential surfaces of the image display units 20a and 20b, respectively.

FIG. 5 is a diagram illustrating a first use state of the wearable device 100 of FIG. 1 according to an embodiment of the disclosure.

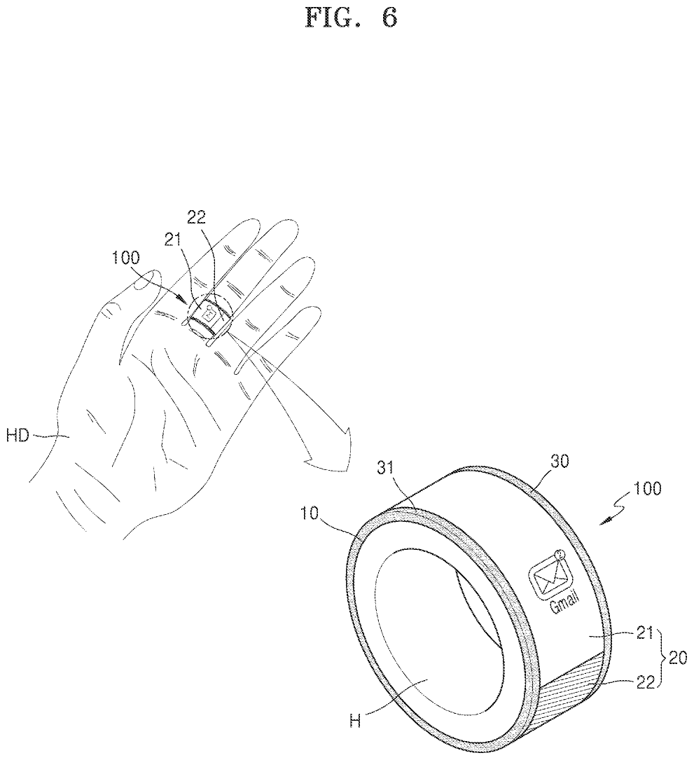

FIG. 6 is a diagram illustrating a second use state of the wearable device 100 of FIG. 1 according to an embodiment of the disclosure.

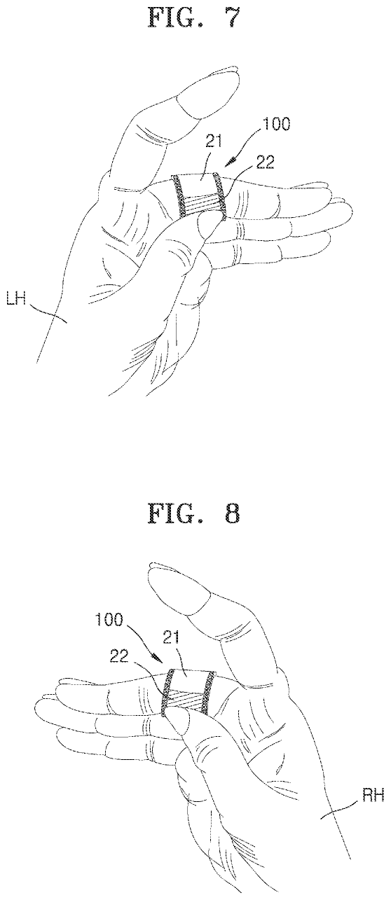

FIG. 7 is a diagram illustrating a third use state of the wearable device 100 of FIG. 1 according to an embodiment of the disclosure.

FIG. 8 is a diagram illustrating a fourth use state of the wearable device 100 of FIG. 1 according to an embodiment of the disclosure.

Referring to FIG. 1, the processor 170 may control an arbitrary region of the image display unit 20 to be the first region 21 and control the remaining region other than the first region 21 to be the second region 22 based on the data related to the motions of the user, measured by the gyroscope sensor 131, the acceleration sensor 132, and the proximity sensor 133 included in the sensing unit 130. For example, the processor 170 may control a region that appears in the sight of the user to be the first region 21 capable of sensing a touch.

A motion of the hand HD of the user on which the wearable device 100 is worn may be sensed by at least one of the gyroscope sensor 131 or the acceleration sensor 132. For example, as illustrated in FIG. 5, a state in which the back of the hand HD faces the eyes of the user may be sensed by the gyroscope sensor 131 and the acceleration sensor 132. This state may be referred to as the first use state of the wearable device 100. In this case, the processor 170 may control a region, that is above the back of the hand HD of the user, of the image display unit 20, to be the first region 21. Referring to FIG. 6, a state in which the palm of the hand HD faces the eyes of the user may be sensed by the gyroscope sensor 131 and the acceleration sensor 132. This state may be referred to as the second use state of the wearable device 100. In this case, the processor 170 may control a region, that is above the palm of the hand HD of the user, of the image display unit 20, to be the first region 21.

Referring to FIG. 7, a state in which the wearable device 100 is worn on a finger of a left hand LH of the user may be sensed by the gyroscope sensor 131 and the acceleration sensor 132. This state may be referred to as the third use state of the wearable device 100. In this case, the processor 170 may appropriately control a region, which appears in the sight of the user, of the image display unit 20, to be the first region 21. Furthermore, as illustrated in FIG. 8, a state in which the wearable device 100 is worn on a finger of a right hand (RH) of the user may be sensed by the gyroscope sensor 131 and the acceleration sensor 132. This state may be referred to as the fourth use state of the wearable device 100. In this case, the processor 170 may appropriately control a region, which appears in the sight of the user, of the image display unit 20, to be the first region 21.

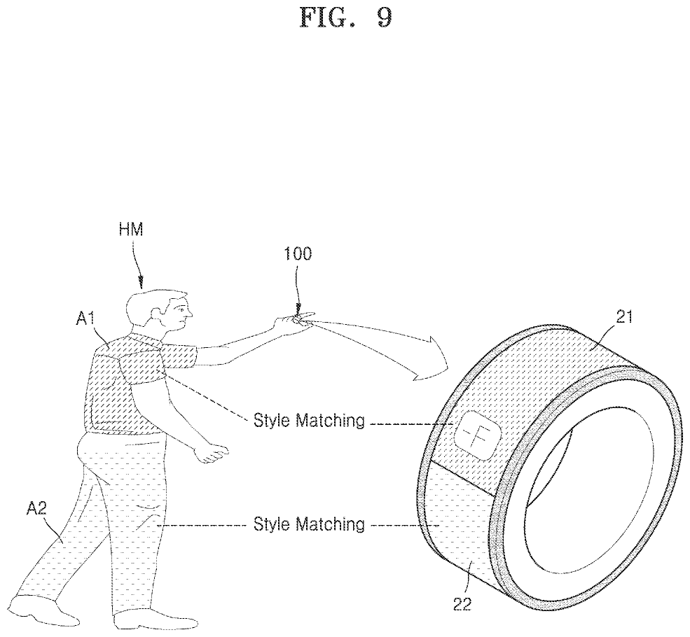

FIG. 9 is a diagram illustrating a style matching function of the wearable device 100 of FIG. 1 according to an embodiment of the disclosure.

Referring to FIG. 9, the first region 21 and the second region 22 included in the wearable device 100 may display different images including different colors, patterns, shapes, and the like, by the processor 170. For example, the first region 21 may be controlled to display a first image including a color, a pattern, a shape, and the like expressed on a top A1 of a user HM wearing the wearable device 100. In addition, the second region 22 may be controlled to display a second image including a color, a pattern, a shape, and the like expressed on a bottom A2 of the user HM wearing the wearable device 100. As described above, the processor 170 may differently display the images on the first region 21 and the second region 22 such that a style according to the clothes worn by the user HM matches the appearance of the wearable device 100. The style matching of the processor 170 may be performed by a certain application installed in a mobile device 200 (see FIG. 17) connected to the wearable device 100 through the wireless communication unit 110. For example, when the user completes a style setting through the application of the mobile device 200, the appearance of the wearable device 100 may be changed to match the style of the user HM.

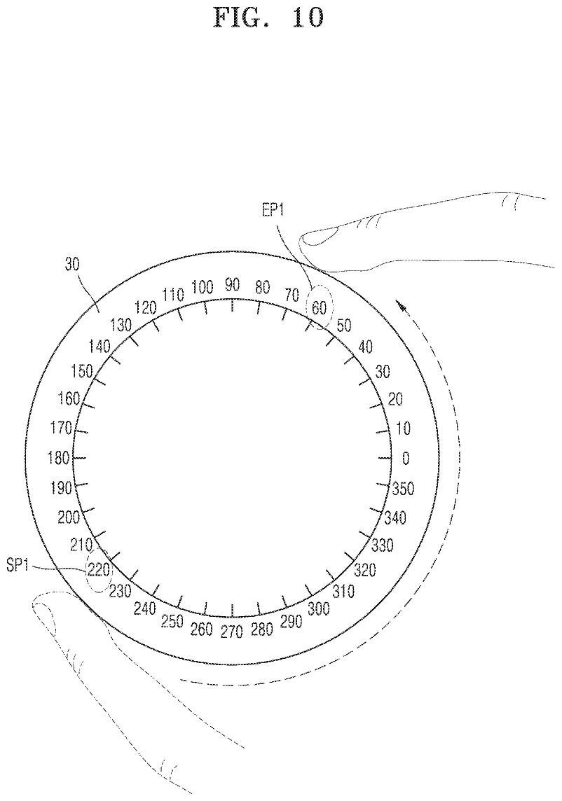

FIG. 10 is a diagram schematically illustrating a use state of the first bezel 30 according to another embodiment of the disclosure, which may be included in the wearable device 100 of FIG. 2 according to an embodiment of the disclosure.

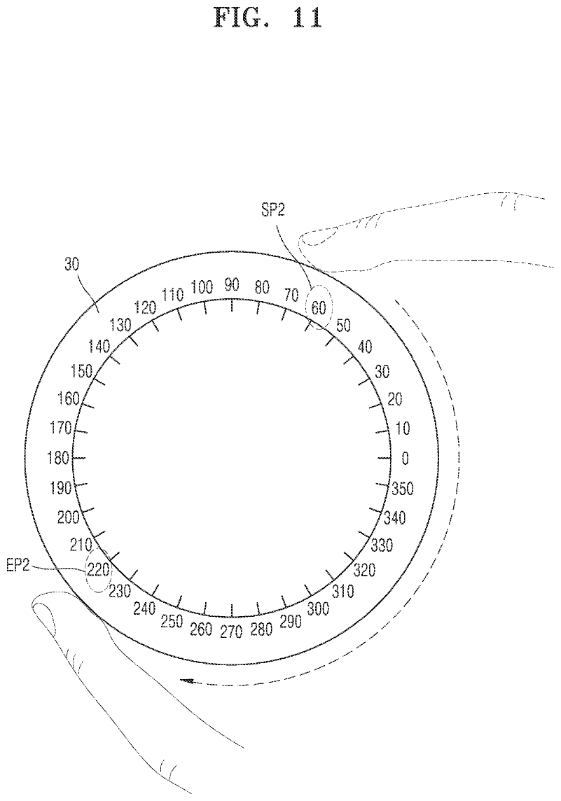

FIG. 11 is a diagram illustrating another use state of the first bezel 30 of FIG. 10 according to an embodiment of the disclosure.

The configuration of the first bezel 30 of FIGS. 10 and 11 may be equally applied to the second bezel 31 of FIG. 2. FIGS. 10 and 11 will be described with reference to the configuration of the body 10 and the image display unit 20 illustrated in FIG. 2.

Referring to FIG. 10, the first bezel 30 may be implemented as an image displayed on both side surfaces of the image display unit 20. The first bezel 30 implemented as the image may be rotatable about the hole H via the touch sensing function of the image display unit 20. For example, the image representing the first bezel 30 may be displayed on both side surfaces of the image display unit 20, and the user may touch and rotate the image of the first bezel 30.

FIG. 10 illustrates the hole H of the body 10 as viewed from above. In this case, gradations for indicating an angle of rotation of the first bezel 30 may be formed on at least one of the top surface or the bottom surface of the body 10. For example, a circular graduated scale indicating 10° per graduation may be formed on the body 10. The user may rotate the first bezel 30 implemented as the image displayed on the image display unit 20 based on the circular graduated scale. For example, as illustrated in FIG. 10, the user may rotate the first bezel 30 from a first start point SP1, which is a point indicated by 220°, to a first end point EP1, which is a point indicated by 60°. In addition, as illustrated in FIG. 11, the user may rotate the first bezel 30 from a second start point SP2, which is a point indicated by 60°, to a second end point EP2, which is a point indicated by 220°. However, the disclosure is not limited thereto, and the user may arbitrarily select any one point among various points displayed on the circular graduated scale as a start point or an end point of the rotation of the first bezel 30. The processor 170 may generate various control signals corresponding to various types of rotation of the first bezel 30 with various start points and end points. Accordingly, the user may generate various control signals for controlling the external electronic device by variously rotating the first bezel 30.

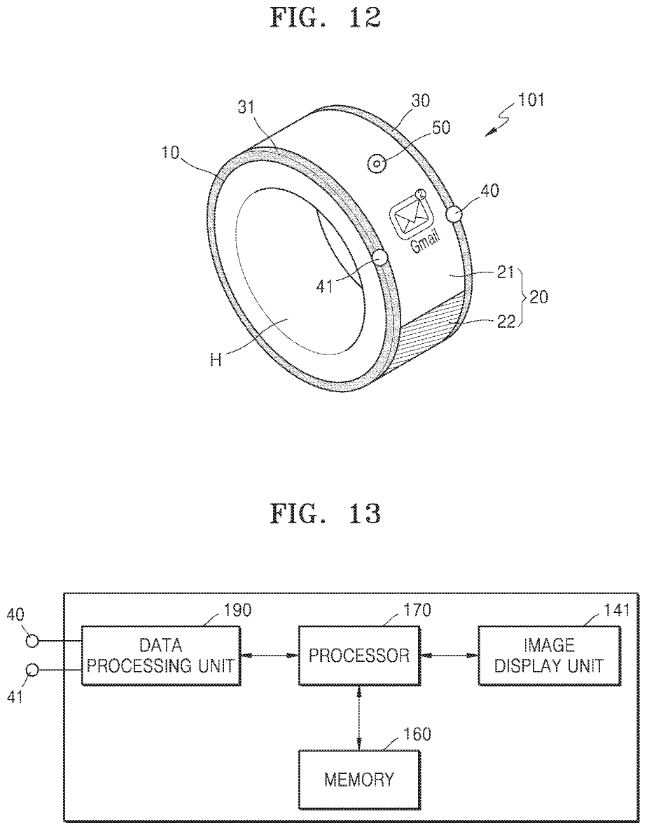

FIG. 12 is a perspective view schematically illustrating a configuration of a wearable device 101 according to an embodiment of the disclosure.

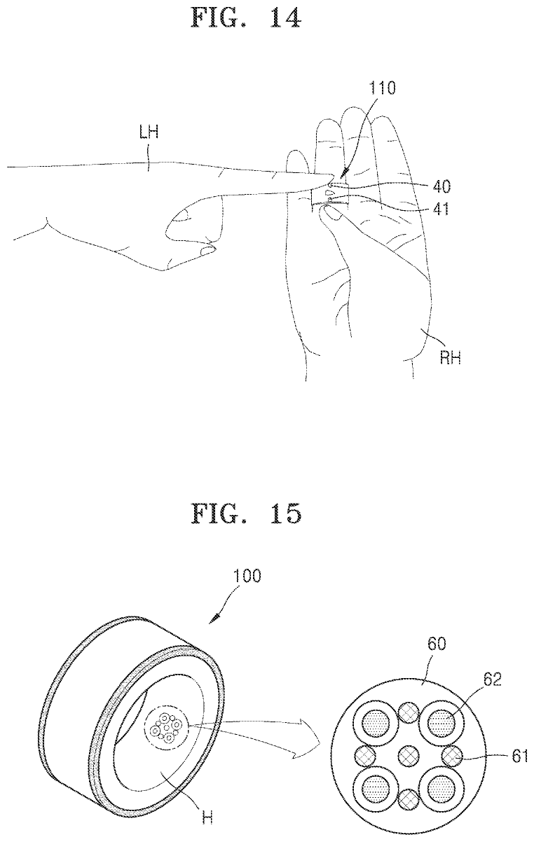

FIG. 13 is a diagram illustrating an electrocardiogram (ECG) measurement system which may be included in the wearable device 101 of FIG. 12. FIG. 14 is a diagram illustrating a use state of the ECG measurement system of FIG. 13 according to an embodiment of the disclosure.

The wearable device 101 of FIG. 12 may be substantially the same as the wearable device 100 of FIG. 2, except that the wearable device 101 further includes a first button 40, a second button 41, and a camera 50. In describing FIG. 12, descriptions that are provided in connection with FIGS. 1 to 3, 4A, 4B, and 5 to 11 will be omitted.

Referring to FIG. 12, the wearable device 101 may further include the first button 40 on the first bezel 30 and the second button 41 on the second bezel 31. The first button 40 and the second button 41 may be mechanical buttons. The user may independently manipulate the first button 40 and the second button 41 to generate different control signals. For example, when the user presses the first button 40, the processor 170 may generate a first control signal, and when the user presses the second button 41, the processor 170 may generate a second control signal that is different from the first control signal. The processor 170 may transmit, to an external electronic device, the first control signal based on the first button 40 and the second control signal based on the second button 41.

For example, the processor 170 may transmit the first control signal based on the first button 40 and the second control signal based on the second button 41, to a mobile device 200 of FIG. 17 through the wireless communication unit 110. A home screen may be displayed on an image display unit of the mobile device 200 by the first control signal. As described above, the first button 40 may perform a home button function with respect to the mobile device 200. Also, a previous screen may be displayed on the image display unit of the mobile device 200 by the second control signal. As described above, the second button 41 may perform a go-back button function with respect to the mobile device 200. However, the disclosure is not limited thereto, and functions of the first button 40 and the second button 41 may be changed according to a setting of the user.

Meanwhile, the wearable device 101 may include the camera 50. The camera 50 may be provided on the image display unit 20. The camera 50 of FIG. 12 may have substantially the same configuration as that of the camera 121 of FIG. 1. A user face recognition function of the camera 50 may be used to unlock the wearable device 101.

Referring to FIG. 13, the wearable device 101 may include the ECG measurement system including the image display unit 141, the memory 160, the processor 170, and a data processing unit 190. The data processing unit 190 may be electrically connected to the first button 40 and the second button 41. In this case, the first button 40 and the second button 41 may function as a first electrode and a second electrode, respectively. As illustrated in FIG. 14, in a state in which a part of the body of the user (e.g., a finger of the left hand (LH)) contacts the first button 40 and another part of the body of the user (e.g., a finger of the right hand (RH)) contacts the second button 41, the processor 170 may control a micro current to flow between the first button 40 and the second button 41. The data processing unit 190 may measure impedance data with respect to the user contacting the first button 40 and the second button 41, and convert the measured impedance data into digital data. The processor 170 may output an electrocardiogram of the user based on data from the data processing unit 190. Data related to the electrocardiogram of the user output by the processor 170 may be stored in the memory 160, and simultaneously, may be provided to the user as an image on the image display unit 141. For example, the data related to the electrocardiogram may be provided to the user as an image in the form of a graph through the image display unit 141.

FIG. 15 is a diagram illustrating a photoplethysmogram sensor 60, which may be included in the wearable device 100 of FIG. 2. In describing FIG. 15, descriptions that are provided in connection with FIGS. 1 to 3, 4A, 4B, and 5 to 14 will be omitted.

Referring to FIG. 15, the wearable device 100 may further include the photoplethysmogram (PPG) sensor 60 on the inner circumferential surface of the body 10 formed by the hole H. The PPG sensor 60 may include a light-emitting device that emits light such as infrared light, visible light, or the like to the skin of the user, and a light-receiving device that receives light reflected by blood vessels of the user. For example, the PPG sensor 60 may include at least one light-emitting diode (LED) 61 that emits infrared light or visible light. In addition, the PPG sensor 60 may include at least one photodiode 62 that receives reflected light.

Referring to FIG. 15, the PPG sensor 60 may include five LEDs 61 arranged in the shape of a cross, and four photodiodes 62 each arranged between the five LEDs 61. However, the disclosure is not limited thereto, and the numbers and arrangement of the LEDs 61 and the photodiodes 62 may be variously determined.

Photoplethysmographic information of the user, obtained by the PPG sensor 60, may be processed by the processor 170, and then stored in the memory 160, while being provided to the user as an image on the image display unit 20.

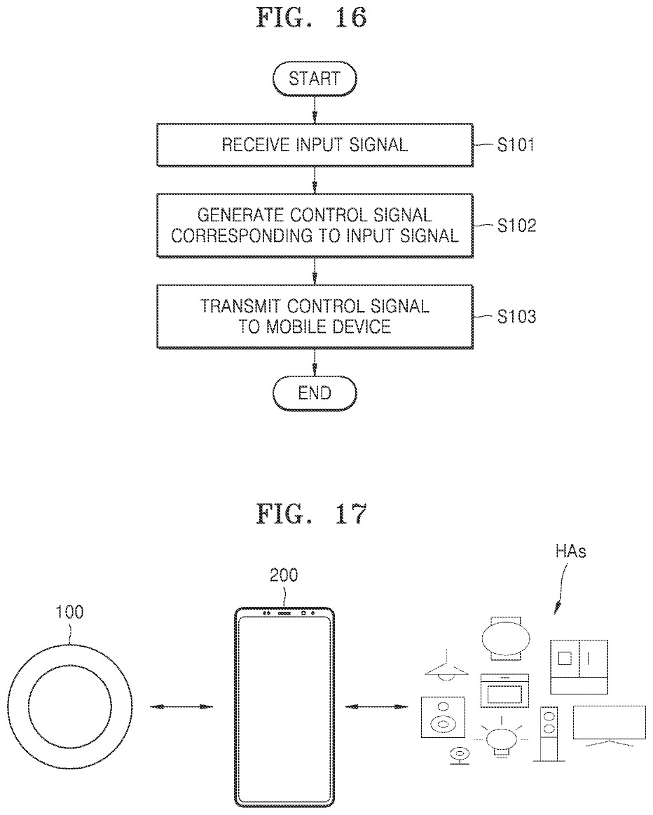

FIG. 16 is a flowchart of a method of generating a signal for controlling an electronic device, according to an embodiment of the disclosure.

FIG. 17 is a diagram schematically illustrating a configuration of an electronic device control system according to an embodiment of the disclosure.

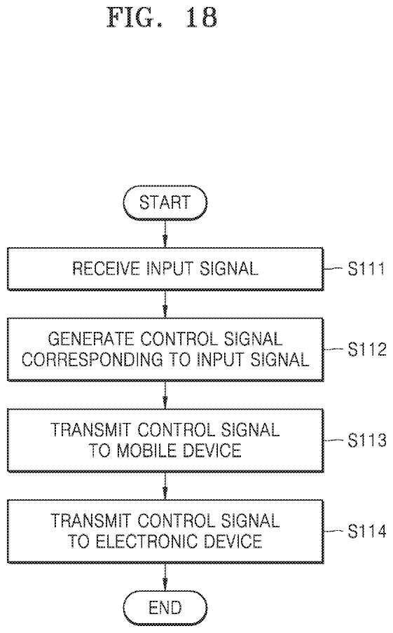

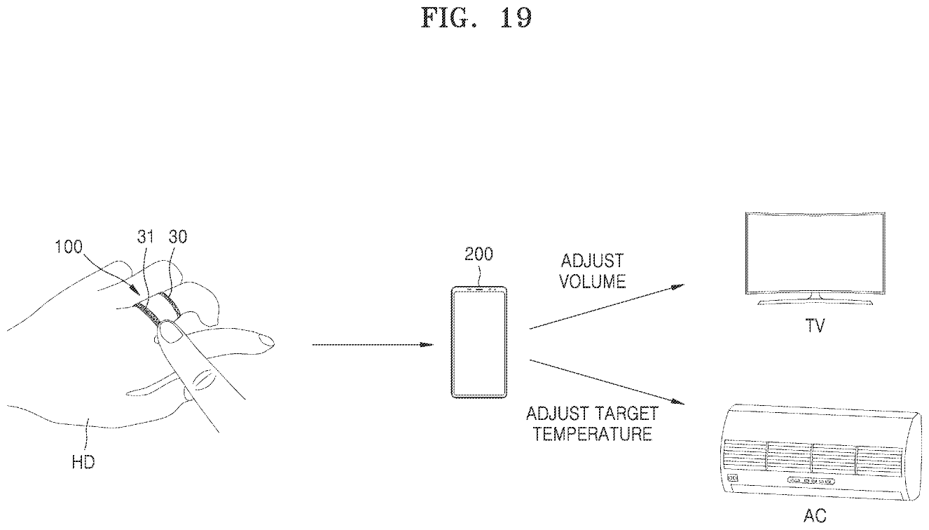

FIG. 18 is a flowchart of a method of controlling the operation of an electronic device, according to an embodiment of the disclosure.

FIG. 19 is a diagram illustrating an operation state of the electronic device control system of FIG. 17 according to an embodiment of the disclosure.

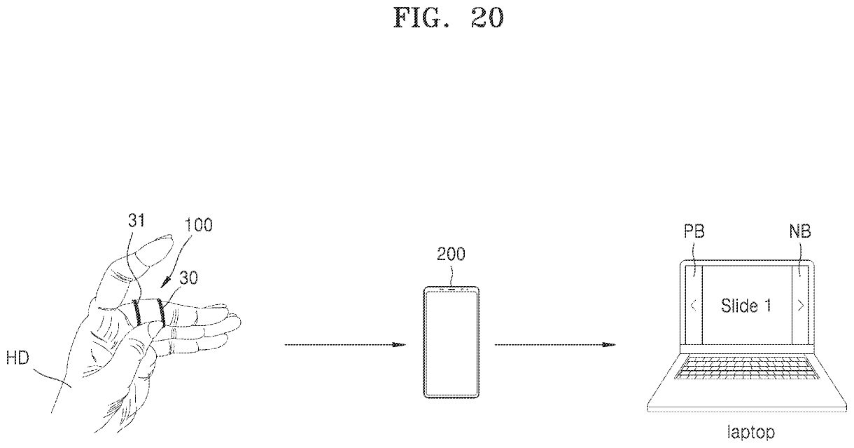

FIG. 20 is a diagram illustrating another operation state of the electronic device control system of FIG. 17 according to an embodiment of the disclosure.

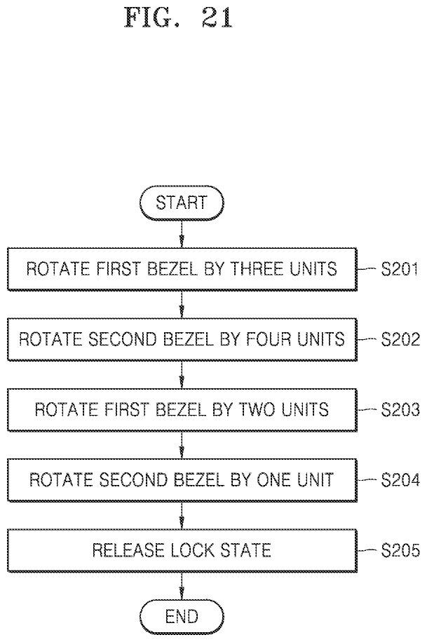

FIG. 21 is a flowchart of a method of unlocking the mobile device 200 of FIG. 17 by using the wearable device 100 of FIG. 2 according to an embodiment of the disclosure.

The wearable device 100 that will be described with reference to FIGS. 16 to 21 may be substantially the same as the wearable device 100 of FIG. 2. FIGS. 16 to 21 will be described with reference to the configuration of the wearable device 100 of FIGS. 1 and 2. Furthermore, the wearable device 101 of FIG. 12 may replace the wearable device 100 that will be described with reference to FIGS. 16 to 21.

Referring to FIG. 16, the method of generating a signal for controlling an electronic device may be implemented by using the wearable device 100 including a first rotation unit and a second rotation unit. The method of generating a signal for controlling an electronic device may include receiving, by the wearable device 100, an input signal according to rotation of the first rotation unit and the second rotation unit at operation S101, generating, by the processor 170 included in the wearable device 100, a control signal corresponding to the input signal at operation S102, and transmitting, by the processor 170, the control signal to the mobile device 200 (see FIG. 17) through the wireless communication unit 110 included in the wearable device 100 at operation S103.

In the receiving, by the wearable device 100, the input signal according to the rotation of the first rotation unit and the second rotation unit at operation S101, the user may apply the input signal to the wearable device 100 by rotating the first rotation unit and the second rotation unit. In this case, the user may apply various input signals to the wearable device 100 via various combinations of orders and angles of rotation of the first rotation unit and the second rotation unit.

For example, the wearable device 100 may include the body 10 of a ring type, and the first bezel 30 and the second bezel 31 provided in the body 10 and configured to rotate independently of each other. The first rotation unit and the second rotation unit included in the wearable device 100 may be the first bezel 30 and the second bezel 31, respectively. In this case, the input signal may be generated by manipulating at least one of the first bezel 30 or the second bezel 31. For example, when the first bezel 30 is rotated by a certain angle in one direction, a first input signal may be generated. In addition, when the second bezel 31 is rotated by a certain angle in one direction, a second input signal that is different from the first input signal may be generated. Furthermore, the user may generate a third input signal that is different from the first input signal and the second input signal by combining an order and angles of rotation of the first bezel 30 and the second bezel 31.

For example, the wearable device 100 may include the body 10 of a ring type and the image display unit 20 having the touch sensing function and surrounding the outer circumferential surface of the body 10. In this case, although not shown in FIG. 16, the method of generating a signal for controlling an electronic device may further include receiving, by the wearable device 100, the input signal according to a touch on the image display unit 20 before the generating, by the processor 170, of the control signal. The input signal according to the touch on the image display unit 20 may be generated by various types of touches with respect to the image display unit 20, including a tap, a hard tap, a double tap, a short touch, a long touch, a multi touch, a drag touch, and a complete cover.

In the generating, by the processor 170 included in the wearable device 100, the control signal corresponding to the input signal at operation S102, the processor 170 may generate control signals corresponding to various input signals input by the user. For example, the processor 170 may generate the control signal corresponding to a combination of an order and angles of rotation of the first rotation unit and the second rotation unit included in the wearable device 100. Here, the first rotation unit and the second rotation unit may correspond to the first bezel 30 and the second bezel 31, respectively.

In addition, for example, the processor 170 may generate a first control signal that corresponds to a first input signal generated by the rotation of the first bezel 30 and a second control signal that is different from the first control signal and corresponds to a second input signal generated by the rotation of the second bezel 31.

In addition, the processor 170 may generate various control signals corresponding to various input signals each generated according to a combination of an order and angles of rotation of the first bezel 30 and the second bezel 31. For example, the processor 170 may generate a third control signal when the first bezel 30 and the second bezel 31 are rotated in the same direction, and may generate a fourth control signal that is different from the third control signal when the first bezel 30 and the second bezel 31 are rotated in opposite directions, respectively.

In addition, the processor 170 may generate various control signals corresponding to various input signals generated according to various types of touches on the image display unit 20.

In the transmitting, by the processor 170, the control signal to the mobile device 200 through the wireless communication unit 110 included in the wearable device 100 at operation S103, the processor 170 may transmit, to the mobile device 200, the control signals corresponding to the various input signals input by the user.

As described above, the processor 170 may transmit, to the mobile device 200, various control signals corresponding to various input signals generated when the user manipulates at least one of the first bezel 30 or the second bezel 31 of the wearable device 100. In addition, the processor 170 may transmit, to the mobile device 200, various control signals corresponding to various types of input signals generated when the user touches the image display unit 20 of the wearable device 100.

Referring to FIG. 17, the electronic device control system may include the wearable device 100 of a ring type including the image display unit 20 having the touch sensing function, and the first bezel 30 and the second bezel 31 provided respectively on both side surfaces of the image display unit 20 to be rotatable, the wearable device 100 of a ring type being configured to generate a control signal according to an input signal received through at least one of the image display unit 20, the first bezel 30, or the second bezel 31, and the mobile device 200 including the processor 170 configured to execute an application for controlling the operation of external electronic devices HAs based on the control signal transmitted from the wearable device 100.

The mobile device 200 may execute the application based on the control signal transmitted from the wearable device 100 to control the operation of the external electronic devices HAs. The mobile device 200 may be for example, a smartphone. However, the disclosure is not limited thereto, and the mobile device 200 may include various types of devices in which an application capable of controlling the operation of the external electronic devices HAs is installed. For example, the mobile device 200 may include a tablet personal computer (PC), a smart watch, and the like.

Referring to FIG. 18, the method of controlling the operation of an electronic device according to an embodiment of the disclosure may include receiving, by the input unit 120 included in the wearable device 100, an input signal at operation S111, generating, by the processor 170 included in the wearable device 100, a control signal corresponding to the input signal at operation S112, transmitting, by the processor 170, the control signal to the mobile device 200 through the wireless communication unit 110 included in the wearable device 100 at operation S113, and controlling the operation of the electronic devices HAs by transmitting the control signal from the mobile device 200 to the electronic devices HAs by an application installed in the mobile device 200 at operation S114. Here, the receiving, by the input unit 120 included in the wearable device 100, the input signal at operation S111, the generating, by the processor 170 included in the wearable device 100, the control signal corresponding to the input signal at operation S112, and the transmitting, by the processor 170, the control signal to the mobile device 200 through the wireless communication unit 110 included in the wearable device 100 at operation S113, may be substantially the same as the receiving the input signal at operation S101, the generating the control signal at operation S102, and the transmitting the control signal to the mobile device 200 at operation S103 illustrated in FIG. 16, respectively. In describing FIG. 18, descriptions that are provided in connection with FIG. 16 will be omitted.

The method of controlling the operation of the electronic devices HAs illustrated in FIG. 18 may be performed by a system for controlling the electronic devices HAs which includes the wearable device 100 of FIG. 17. Accordingly, the user may control the operation of the electronic devices HAs by using the wearable device 100.

In the receiving, by the input unit 120 included in the wearable device 100, the input signal at operation S111, the user may apply the input signal to the wearable device 100 through the input unit 120. For example, the user may apply the input signal to the wearable device 100 through the first bezel 30, the second bezel 31, and the image display unit 20 included in the wearable device 100.

In the controlling the operation of the electronic devices HAs at operation S114, the control signal may be transmitted from the mobile device 200 to the electronic devices HAs, and the operation of the electronic devices HAs may be controlled by the control signal. For example, various types of control signals may be transmitted from the wearable device 100 to the mobile device 200. An application capable of processing the control signal received from the wearable device 100 may be installed in the mobile device 200. For example, the mobile device 200 may be a smartphone. However, the disclosure is not limited thereto, and the mobile device 200 may include various types of devices in which the application capable of processing the received control signal may be installed. The application installed in the mobile device 200 may transmit various control signals received by the mobile device 200 to the electronic devices HAs. The electronic devices HAs may perform various operations according to the received various control signals. For example, a first operation state of the electronic devices HAs according to the first control signal and a second operation state of the electronic devices HAs according to the second control signal may be different from each other.

Referring to FIG. 19, the user wearing the wearable device 100 on the hand HD may set a control target to a television (TV) from among the external electronic devices HAs by using the touch screen implemented by the image display unit 20. In this case, the user may adjust the volume of the TV by rotating the first bezel 30. For example, when the user rotates the first bezel 30 clockwise, a control signal for increasing the volume of the TV may be transmitted to the TV via the application of the mobile device 200, and thus, the volume of the TV may be increased. In addition, when the user rotates the first bezel 30 counterclockwise, a control signal for decreasing the volume of the TV may be transmitted to the TV via the application of the mobile device 200, and thus, the volume of the TV may be decreased. In addition, the user may change the channel of the TV by rotating the second bezel 31 in a state in which the control target is set to the TV.

Referring to FIG. 19, the user wearing the wearable device 100 on the hand HD may set the control target to an air conditioner (AC) from among the external electronic devices HAs by using the touch screen implemented by the image display unit 20. In this case, the user may adjust the target temperature of the AC by rotating the first bezel 30. For example, when the user rotates the first bezel 30 clockwise, a control signal for increasing the target temperature of the AC may be transmitted to the AC through the application of the mobile device 200, and thus, the target temperature of the AC may be increased. In addition, when the user rotates the first bezel 30 counterclockwise, a control signal for decreasing the target temperature of the AC may be transmitted to the AC through the application of the mobile device 200, and thus, the target temperature of the AC may be decreased. Furthermore, the user may adjust the fan speed of the AC by rotating the second bezel 31 in a state in which the control target is set to AC.

Referring to FIG. 20, the user wearing the wearable device 100 on the hand HD may set the control target to a laptop from among the external electronic devices HAs by using the touch screen implemented by the image display unit 20. The user may perform various tasks such as word processing on PowerPoint or Internet surfing, by using the laptop. For example, functions of the first bezel 30 and the second bezel 31 may be set such that, while performing a certain task on a PowerPoint document, the user may activate a go-to-previous-slide function by rotating the first bezel 30, and activate a go-to-next-slide function by rotating the second bezel 31. As described above, with respect to a PowerPoint document, the first bezel 30 may be set to have a function of a go-back button PB, and the second bezel 31 may be set to have a function of a go-forward button NB. The functions of the first bezel 30 and the second bezel 31 may be changed according to a setting of the application of the mobile device 200.

Referring to FIGS. 19 and 20, the functions of the first bezel 30 and the second bezel 31 may be variously changed according to settings of the application of the mobile device 200. In addition, various types of control may be performed on the external electronic devices HAs according to combinations of rotation of the first bezel 30 and the second bezel 31. For example, when the user rotates both the first bezel 30 and the second bezel 31 clockwise, the volume of the TV may be increased and the target temperature of the AC may be increased. On the contrary, when the user rotates both the first bezel 30 and the second bezel 31 counterclockwise, the volume of the TV may be decreased and the target temperature of the AC may be decreased.

Furthermore, various types of control may be performed on the mobile device 200 according to combinations of orders and angles of rotation of the first bezel 30 and the second bezel 31. For example, the mobile device 200 may be unlocked by appropriately adjusting an order and angles of rotation of the first bezel 30 and the second bezel 31. One full rotation of each of the first bezel 30 and the second bezel 31 may be divided into n units of angle, so as to express an angle of rotation of the first bezel 30 or the second bezel 31 as a multiple of the unit. In this case, a pattern for unlocking the mobile device 200 may be produced by appropriately combining an order and angles of rotation of the first bezel 30 and the second bezel 31.

Referring to FIG. 21, the method of unlocking the mobile device 200 by using the first bezel 30 and the second bezel 31 may include rotating the first bezel 30 by three units at operation S201, rotating the second bezel 31 by four units at operation S202, rotating the first bezel 30 by two units at operation S203, rotating the second bezel 31 by one unit at operation S204, and releasing a lock state at operation S205.

However, the disclosure is not limited thereto, and the order and angles of rotation of the first bezel 30 and the second bezel 31 for producing the pattern for unlocking the mobile device 200 may be variously changed according to a setting of the user.

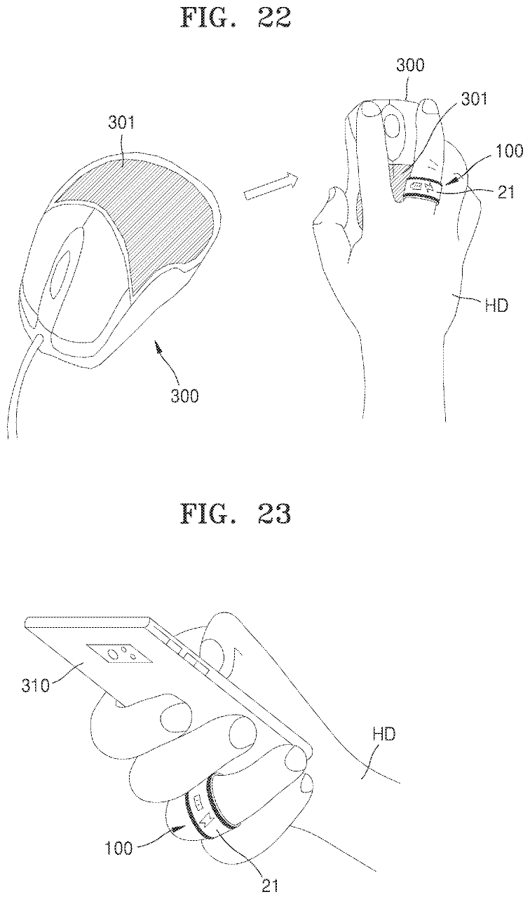

FIG. 22 is a diagram illustrating a method of performing wireless charging of the wearable device 100 of FIG. 17 according to an embodiment of the disclosure. FIG. 23 is a diagram illustrating a method of performing wireless charging of the wearable device 100 of FIG. 17 according to an embodiment of the disclosure.

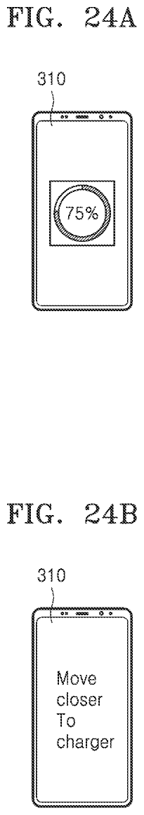

FIGS. 24A and 24B are diagrams illustrating an operation of the mobile device 310 of FIG. 23 while performing wireless charging of the wearable device 100 of FIG. 23 according to various embodiments of the disclosure.

Referring to FIG. 22, the wearable device 100 may be configured to be rechargeable by using a wireless charging device mounted on an external electronic device. For example, a mouse 300, which is an external electronic device, may include a charging pad 301 which is a wireless charging device. When the user wearing the wearable device 100 on the hand HD holds and uses the mouse 300, the wearable device 100 and the charging pad 301 may be sufficiently close to each other to perform wireless charging. In this case, wireless charging of the wearable device 100 may be performed, and the state of charge of the wearable device 100 may be displayed on the first region 21 of the image display unit 20 of the wearable device 100.

Referring to FIG. 23, for example, a built-in wireless charging device may be included in a mobile device 310 which is an external electronic device. The mobile device 310 may include a smartphone. When the user wearing the wearable device 100 on the hand HD holds and uses the mobile device 310, the wearable device 100 and the mobile device 310 may be sufficiently close to each other to perform wireless charging. In this case, wireless charging of the wearable device 100 may be performed, and the state of charge of the wearable device 100 may be displayed on the first region 21 of the image display unit 20 of the wearable device 100. Also, as illustrated in FIG. 24A, while the wearable device 100 is being charged by the mobile device 310, the state of charge of the wearable device 100 may be displayed as an image on an image display unit included in the mobile device 310. Accordingly, the user may conveniently check the state of charge of the wearable device 100 through the image displayed on the wearable device 100 or the mobile device 310. Furthermore, as illustrated in FIG. 24B, when the wireless charging becomes unavailable as the distance between the wearable device 100 and the mobile device 310 is increased, a warning message for the user may be displayed on the image display unit included in the mobile device 310. For example, when the wireless charging is unavailable due to a long distance between the wearable device 100 and the mobile device 310, a warning message to inform the user that the distance between the two devices needs to be closer for the wireless charging. The content of the warning message may be variously modified according to a setting of the application of the mobile device 310.

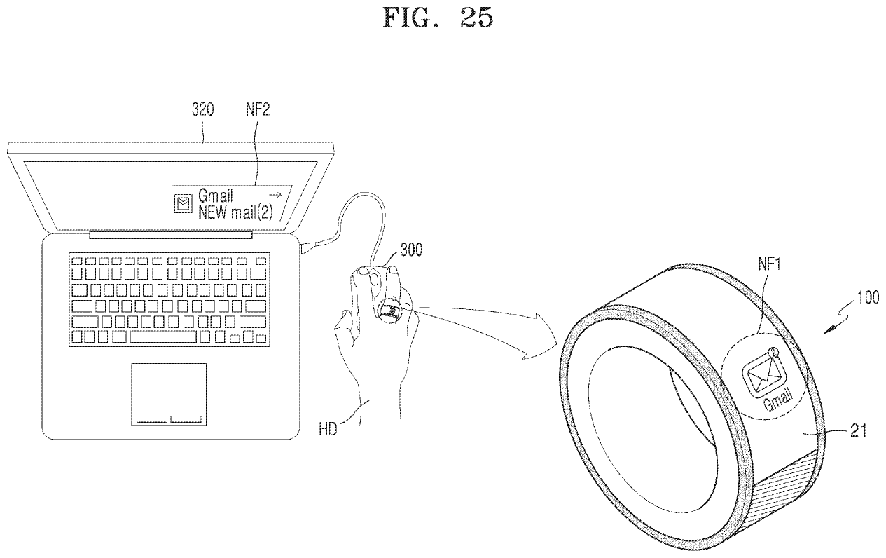

FIG. 25 is a diagram illustrating an interaction between the wearable device 100 and an external electronic device 320 that occurs while performing wireless charging of the wearable device 100 of FIG. 17 according to an embodiment of the disclosure.

Referring to FIG. 25, when the user wearing the wearable device 100 on the hand HD uses the mouse 300 connected to the external electronic device 320, the wearable device 100 may be wirelessly charged by a wireless charging device included in the mouse 300. Here, the external electronic device 320 may be a laptop.

The wearable device 100 may be configured to transmit and receive data to and from the external electronic device 320 through the wireless communication unit 110 (see FIG. 1) while the wireless charging is being performed by the wireless charging device included in the mouse 300. For example, the short-range communication module 111 (see FIG. 1) included in the wearable device 100 and having an NFC function may interact with the mouse 300, and thus a channel for transmitting and receiving data may be established between the wearable device 100 and the external electronic device 320. Referring to FIG. 25, data related to a first notification image NF1 displayed on the first region 21 included in the image display unit 20 of the wearable device 100 may be transmitted to the external electronic device 320. Thereafter, a second notification image NF2 corresponding to the first notification image NF1 may be displayed on an image display unit of the external electronic device 320.

As described above, while the wireless charging of the wearable device 100 is being performed, data transmission and reception between the wearable device 100 and the external electronic device 320 may be available, and thus, more convenient and diverse user interface environments may be created.

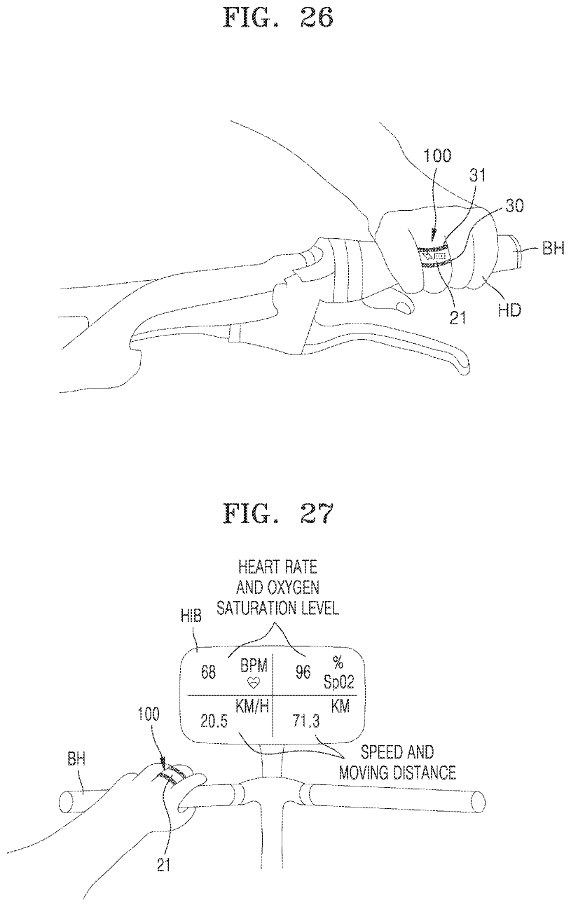

FIG. 26 is a diagram illustrating a method of performing wireless charging of the wearable device 100 of FIG. 17 according to an embodiment of the disclosure.

FIG. 27 is a diagram illustrating an interaction between the wearable device 100 and an external electronic device HIB that occurs while performing wireless charging of the wearable device 100 of FIG. 26 according to an embodiment of the disclosure.

Referring to FIG. 26, when the user wearing the wearable device 100 on the hand HD holds a handle BH of a bicycle or motorcycle, the wearable device 100 may be wirelessly charged by a wireless charging device included in the handle BH. The wireless charging device having a coil structure for providing power to the wearable device 100 may be embedded in the handle BH. However, the disclosure is not limited thereto, and the type of the wireless charging device embedded in the handle BH may vary. When the wireless charging of the wearable device 100 is performed by the wireless charging device of the handle BH, the state of charge of the wearable device 100 may be displayed on the first region 21 of the image display unit 20 of the wearable device 100.

Referring to FIG. 27, the wearable device 100 may be configured to transmit and receive data to and from the external electronic device HIB mounted on the bicycle or motorcycle through the wireless communication unit 110 (see FIG. 1) while the wireless charging is being performed by the wireless charging device included in the handle BH. In this case, the external electronic device HIB may be an electronic health-information board configured to measure health information-related data of the user (e.g., a heart rate, an oxygen saturation level, a moving speed and a moving distance of the user, and the like.) and provide the health information-related data to the user as an image.

For example, the short-range communication module 111 (see FIG. 1) included in the wearable device 100 and having the NFC function may interact with the handle BH, and thus a channel for transmitting and receiving data may be established between the wearable device 100 and the external electronic device HIB. Accordingly, various pieces of health information-related data displayed on the external electronic device HIB may be transmitted to the wearable device 100. Thereafter, the health information-related data may be displayed on the first region 21 of the image display unit 20 of the wearable device 100.

According to an example embodiment of the disclosure, a wearable device of a ring type capable of controlling various electronic devices through an application installed in a mobile device may be provided.

According to an example embodiment of the disclosure, an electronic device may be more conveniently controlled by using the wearable device of a ring type including an image display unit that surrounds the outer circumferential surface thereof and has a touch sensing function, and a bezel on both side surfaces of the image display unit. For example, a user may easily provide an input signal to the wearable device by touching the image display unit or rotating the bezel, so as to generate a control signal for more conveniently controlling the electronic device.

According to an example embodiment of the disclosure, a method of controlling the operation of an electronic device by using a wearable device of a ring type configured to generate a control signal for controlling an electronic device according to an input of a user, and a mobile device including an application for controlling the operation of the electronic device based on the control signal from the wearable device may be provided.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable device comprising:
a ring-shaped body comprising a hole;
an image display unit surrounding an outer circumferential surface of the ring-shaped body;
a first ring-shaped bezel and a second ring-shaped bezel respectively provided on a first side surface and a second side surface of the image display unit to be independently rotatable about the hole;
a sensor configured to measure a motion;
a processor configured to:
control a first region of the image display unit to have a touch sensing function, and
control a second region that is different from the first region not to have the touch sensing function; and
a transceiver unit configured to transmit, to an external electronic device, a control signal generated by the processor according to an input signal by a touch on the image display unit or an input signal by rotation of at least one of the first ring-shaped bezel or the second ring-shaped bezel.

2. The wearable device of claim 1, wherein the processor is further configured to:
control an arbitrary region of the image display unit to be the first region; and
control a remaining region other than the first region to be the second region based on data related to a motion measured by the sensor.

3. The wearable device of claim 1, wherein the first region occupies about 40% to about 50% of an area of an outer circumferential surface of the image display unit.

4. The wearable device of claim 1, wherein the sensor comprises at least one of a gyroscope sensor or an acceleration sensor.

5. The wearable device of claim 1, wherein the processor is further configured to:
generate a first control signal when the first ring-shaped bezel and the second ring-shaped bezel are rotated in a same direction,
generate a second control signal that is different from the first control signal when the first ring-shaped bezel and the second ring-shaped bezel are rotated in opposite directions respectively, and
transmit the first control signal and the second control signal to the external electronic device through the transceiver unit.

6. The wearable device of claim 1, wherein the processor is further configured to:
generate a control signal corresponding to a combination of an order and angles of rotation of the first ring-shaped bezel and the second ring-shaped bezel, and
transmit the control signal to the external electronic device through the transceiver unit.

7. The wearable device of claim 1, wherein the processor is further configured to switch functions of the first ring-shaped bezel and the second ring-shaped bezel with each other based on data related to positions of the first ring-shaped bezel and the second ring-shaped bezel measured by the sensor.

8. The wearable device of claim 1, wherein the processor is further configured to transmit, to the external electronic device, a control signal according to a touch on the first region of the image display unit, through the transceiver unit.

9. The wearable device of claim 1,
wherein the first ring-shaped bezel and the second ring-shaped bezel are implemented as images formed on both side surfaces of the image display unit, respectively, and
wherein the first ring-shaped bezel and the second ring-shaped bezel implemented as the images are rotatable about the hole via the touch sensing function of the image display unit.

10. The wearable device of claim 1, further comprising:
a first button on the first ring-shaped bezel and a second button on the second ring-shaped bezel,
wherein the processor is further configured to transmit, to the external electronic device, a first control signal based on the first button and a second control signal, which is different from the first control signal, based on the second button.

11. The wearable device of claim 1, further comprising a first electrode on the first ring-shaped bezel and a second electrode on the second ring-shaped bezel.

12. The wearable device of claim 11, further comprising:
a data processing unit configured to process an electrical signal from the first electrode and the second electrode,
wherein the processor is further configured to output an electrocardiogram (ECG) based on data from the data processing unit.

13. The wearable device of claim 1, further comprising a photoplethysmogram (PPG) sensor on an inner circumferential surface of the ring-shaped body.

14. The wearable device of claim 1,
wherein the wearable device is configured to be rechargeable by using an external wireless charging device, and
wherein the wearable device is further configured to transmit and receive data to and from the external electronic device through the transceiver unit while charging of the wearable device is being performed by the external wireless charging device.
